(19) United States
(12) Patent Application Publication
(10) Pub. No.: US 2020/0205685 A1
(43) Pub. Date: Jul. 2, 2020

(54) Home Kit for Diagnosis of Intolerance for Animals and Humans

(71) Applicant: AFFORDABLE ALLERGY TEST, LLC, Lawrenceville, GA (US)

(72) Inventor: Lisa Blaurock, Lawrenceville, GA (US)

(73) Assignee: AFFORDABLE ALLERGY TEST, LLC, Lawrenceville, GA (US)

(21) Appl. No.: 16/723,367

(22) Filed: Dec. 20, 2019

Related U.S. Application Data
(60) Provisional application No. 62/784,835, filed on Dec. 26, 2018.

(51) Int. Cl. A61B 5/04; G01R 33/20; A61K 41/00

(52) U.S. Cl. CPC A61B 5/04; G01N 1/28; A61K 41/0004; G01R 33/20

(57) ABSTRACT

A method measures intolerances in a subject to various factors, such as food, beverage, metals and minerals, nutrients, and environmental factors using a testing machine that measures bioresonance, radionics, and/or quantum response.

This application claims priority under 35 U.S.C. § 119 to U.S. Provisional App. No. 62/784,835, filed 26 Dec. 2019, the entirety of which is incorporated by reference herein.

### BACKGROUND

### Field of Endeavor

The present invention relates to processes useful for diagnosing food and environmental intolerances and deficiencies, and more specifically to a process for analyzing hair samples to determine food, nutritional, environmental, metal, and/or mineral intolerances in a subject.

### Brief Description of the Related Art

Food and environmental intolerances are non-IgE mediated hypersensitivities or non-allergic hypersensitivities, and can result in difficulty in digesting certain foods and other health-related issues. Food and environmental intolerances are distinct from food and environmental allergies, which actually trigger the immune system and produce a histamine response, while food and environmental intolerances do not.

Symptoms from IgE-mediated allergic reactions are distinct from symptoms from food and environmental intolerances. Specifically, allergy symptoms include swelling of the lips, face, and tongue, hives, itching, nausea, vomiting, tightness in the chest, wheezing, and abdominal pain. Intolerance symptoms include headache, mood swings, eye mucus, runny nose, bloating, IBS, water retention, diarrhea, joint pain, skin irritation, itching, and acne. Allergic reactions are immediate and can be life-threatening; whereas, intolerances then to be delayed reactions and are not life-threatening.

To manage the symptoms of food and environmental intolerances, knowledge of the triggers of the intolerances is essential; however, these can be different for each subject. Every being, whether a human or animal, is a vibrating system and has a natural frequency. The natural frequency is the frequency observed when no driving or suppressing forces are acting on the system. Resonance exists when the system responds to an alternative driving force with maximum amplitude, and the frequency of the driving force matches the natural (non-damped) frequency of the system. A biological system will respond in a measurable manner only to those exogenous oscillations, that is, the alternate driving force, that match the natural endogenous electromagnetic (“EM”) frequency. Bio-resonance technology allows for testing for food and environmental intolerances using only a small sample from a subject, such as a few strands of hair, and can provide the subject with valuable information that is helpful in managing symptoms resulting from the intolerance.

### SUMMARY

A method measures intolerances in a subject to various factors such as food, beverage, metals and minerals, nutrients, and environmental factors using a testing machine that measures bioresonance, radionics, and/or quantum response.

Still other aspects, features, and attendant advantages of the present invention will become apparent to those skilled in the art from a reading of the following detailed description of embodiments constructed in accordance therewith, taken in conjunction with the accompanying drawings.

### BRIEF DESCRIPTION OF THE DRAWINGS

The invention of the present application will now be described in more detail with reference to exemplary embodiments of the apparatus and method, given only by way of example, and with reference to the accompanying drawings.

FIG. 1 illustrates a flow chart of an exemplary process as described herein.

FIG. 2 illustrates exemplary results of the method as described herein.

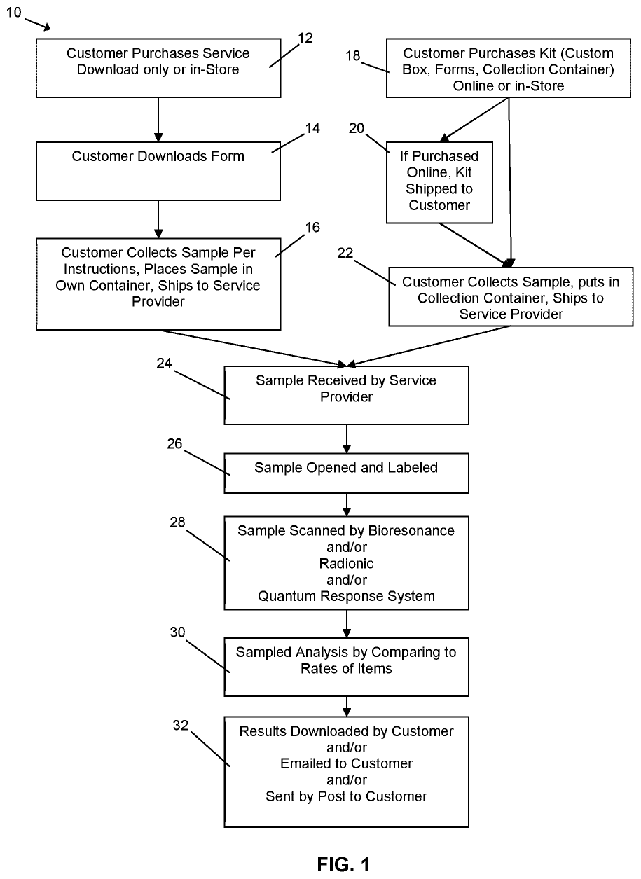

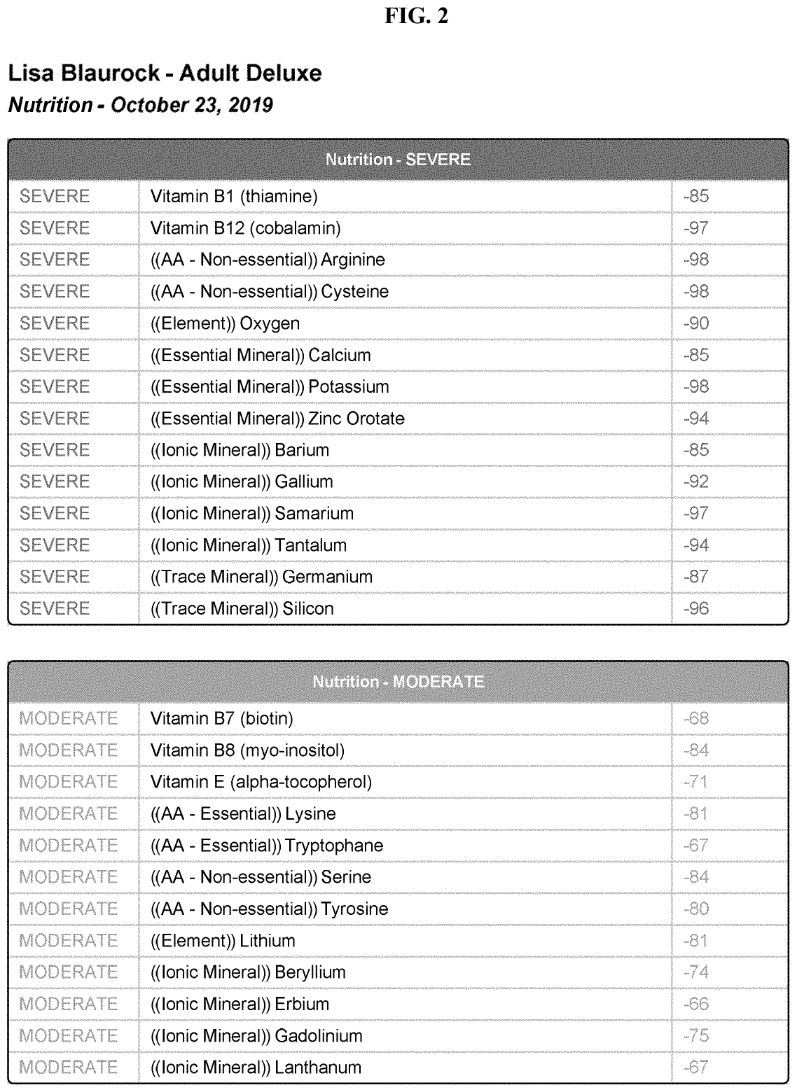

### DETAILED DESCRIPTION OF EXEMPLARY EMBODIMENTS

Referring to the drawing figures, like reference numerals designate identical or corresponding elements throughout the several figures.

The singular forms “a,” “an,” and “the” include plural referents unless the context clearly dictates otherwise. Thus, for example, reference to “a solvent” includes reference to one or more of such solvents, and reference to “the dispersant” includes reference to one or more of such dispersants.

Concentrations, amounts, and other numerical data may be presented herein in a range format. It is to be understood that such range format is used merely for convenience and brevity and should be interpreted flexibly to include not only the numerical values explicitly recited as the limits of the range, but also to include all the individual numerical values or sub-ranges encompassed within that range as if each numerical value and sub-range is explicitly recited.

For example, a range of 1 to 5 should be interpreted to include not only the explicitly recited limits of 1 and 5, but also to include individual values such as 2, 2.7, 3.6, 4.2, and sub-ranges such as 1-2.5, 1.8-3.2, 2.6-4.9, etc. This interpretation should apply regardless of the breadth of the range or the characteristic being described, and also applies to open-ended ranges reciting only one end point, such as “greater than 25,” or “less than 10.”

Generally, the methods described herein are processes that use the theory and technology of bioresonance and energy medicine to measure for one or more intolerances in a subject. Products are also described herein including a kit that can include collection containers, product box, and instruction form with an information sheet.

With general reference to the single drawing figure, an exemplary process (10) begins with the subject either obtaining a kit from an online or “brick and mortar” retailer, or downloading a form and instructions (12, 14, 18). The sample is collected from the subject, placed in a container, and shipped to the service provider (16, 22), who receives it (24), opens and labels it (26), and scans it using a bioresonance, radionic, or quantum response system (28). The results of the scan are analyzed by comparing to rates of items (30), and either downloaded by the subject, emailed to the subject or sent by post to the subject (32).

The kit as described herein can be purchased two ways: via a downloadable form (12) or in retail (online) stores (18). If purchased via a download, the subject downloads the form and instructions (14). The subject collects a sample, e.g., one or more hairs, according to the downloadable instructions, puts the sample into a container provided by the subject, and ships the container containing the sample back to the service provider (16). If purchased via online or at a retail “brick and mortar” establishment, the subject obtains a kit from the online retailer (20) or a “brick and mortar” store (18). A “brick and mortar” store is one that has a physical place of business and serves customers at that place of business. In this case, the subject collects the sample according to the provided instructions and places the collected sample into the provided collection container and ships to the service provider (22).

The collected sample, whether contained in the provided collection container or one provided by the subject, is received by the service provider (24), opened and labeled by the service provider (26).

The sample is scanned using a Bioresonance and/or Radionic and/or Quantum response system (28). The results of the scan are analyzed by comparing the sample to the rates of items (30). The results are downloaded from the system and provided to the subject (32).

An exemplary system as used herein that is employed to do the analysis of the sample uses the principles of bioresonance. These principles rely on the discovery that all three levels of biological organization, that is, organismal, cellular, and molecular, emit metabolic energy in the form of electromagnetic fields (“EMF”). In the human organism, for example, an average EMF is emitted at a frequency of about 7.8 Hz; however, every cell, tissue, organ, nerve, artery, and even genes emit EMF at differing frequencies. When a sample from a subject is analyzed and the EMF is found to differ from the normal frequency, intolerances to both food and environmental influences can result. Often these intolerances are indicative of the presence of certain disease states and can be detected using EMF technology before disease symptoms manifest.

The sample can be any small part of the subject that can be excised, cut, scraped, pulled, or otherwise collected from the subject. For example, the sample can be strands of the subject's hair or fur. Several strands of hair or fur can be collected, such as 20, 15, 10, 9, 8, 7, 6, 5, 4, or 3 strands of hair or fur.

The subject can be any organism that produces hair or fur. For example, the subject can be a human, dog, cat, horse, or other domesticated or non-domesticated animal that produces hair or fur. The hair or fur collected for the sample can come from any part of the subject's body, including but not limited to, the head, the arms, the legs, the nose, the face, or the genital area. The hair or fur can be collected without regard for what the subject has recently ingested.

The collection container can be any container that can contain the sample advantageously without contamination. For example, if provided by the subject, any plastic, metal, or any other material is sufficient, as long as it has an enclosure such as lid that is able to completely contain the sample during shipping. A reusable container that is typically used for food storage is one example.

The collection container provided with the kit as described herein can also be any container that can completely contain the sample, particularly during shipping.

Once the service provider receives the sample from the subject, the subject's information from the testing form provided with the kit is entered into a Customer Relationship Managment (“CRM”) system. The information from the testing form can include, but is not limited to, gender, date of birth, home address and email address, etc. The information is for identification purposes only. Once the subject is set up in the CRM, the test type is chosen.

Test types include bioreasonance, which measures food intolerances and nutritional deficiencies, metal and mineral analysis, and environmental analysis. If the subject is a domestic animal, such as a dog or cat, tolerance for certain animal ‘treats’ can be tested. One or all of these can be run depending on the desires of the subject.

The sample is removed from the container, either the container provided by subject or from the purchased kit, and the sample is placed in a parchment bag. The parchment bag containing the sample is placed into the input cup or plate on the testing machine.

The testing machine can be any machine that can measure bioresonance, that is, the driving frequency of the sample, and compares this frequency to the natural frequency for the type of subject (human, animal), which can also be referred to as the resonance frequency. The friction surface of the input cup or plate acts as the resonance detector. Examples of the testing machine include the Marslll Multiple Analytical Resonance System from Bruce Copen Laboratories (Munich, Germany). Sensitiv Imago, Medicomat −36 7D-NLS, Melatron Hunter 4025, JiaHao Bioreasonance TherapyDevices 3D NLS Health analyzer, Biocom Optima, Biocom Body Check, Mantra !, Quantum Care II, Care ND, etc.

The sample may have blocked zones which cannot be measured. When this occurs, the testing machine can detect these zones using the VIBA generator, also known as a “vast interactive bio-system analyser” (Bruce Copen Labs), optionally with the PEAR program (see pearlab.icrl.org). The VIBA generator allows radionic analyses to be carried out, which makes it possible to balance the subject's energy deficit and remove interference fields by identifying parameters in the analysis and modulate the frequency for the particular subject. A color tune can also be performed to unblock zones in the sample. This encompasses color and musical tone therapy which allows for virtual broadcasting of blocked body zones by means of tones and colors.

The testing machine calculates the results from the sample, and compares the results from the sample to known frequencies for the items being measured. The degree of comparative resonance determines the intolerances and deficiencies for those items. The results are saved and synced to the subject's CRM.

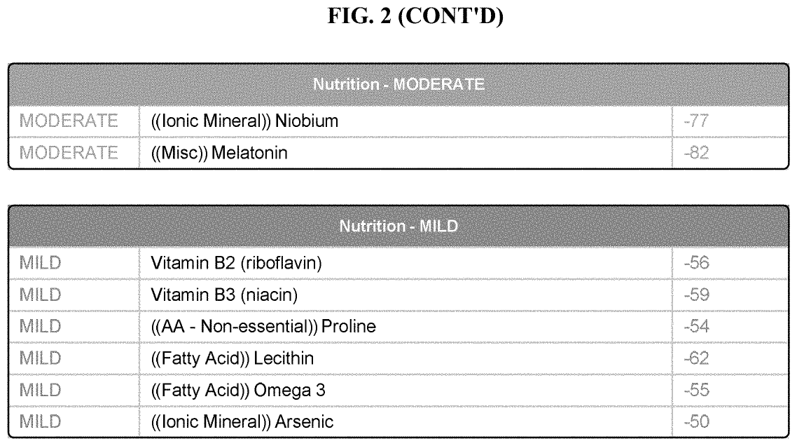

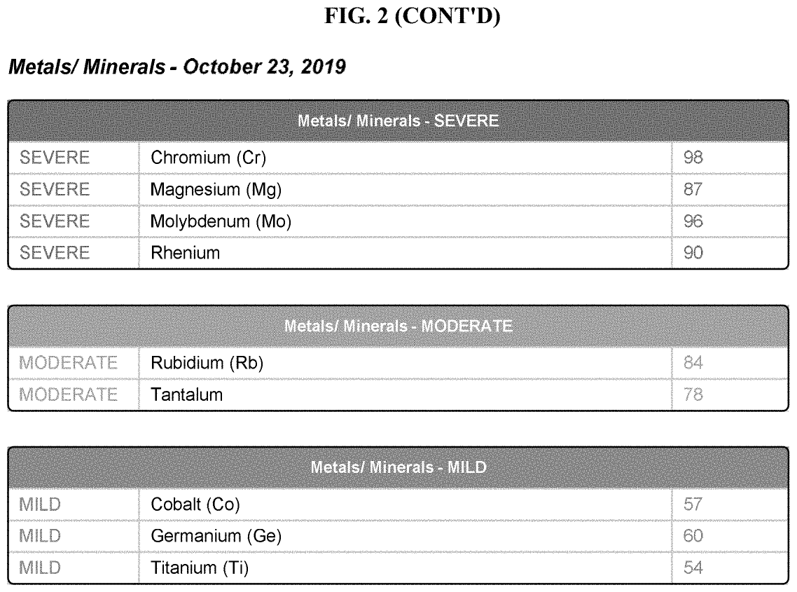

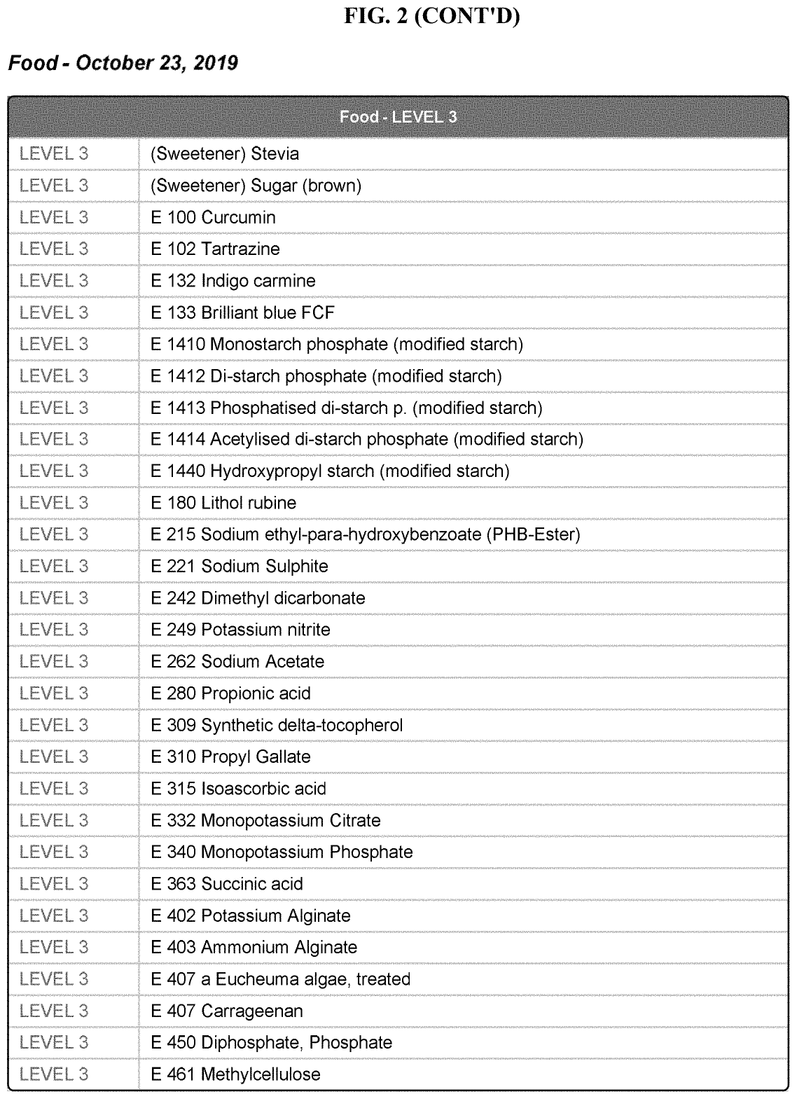

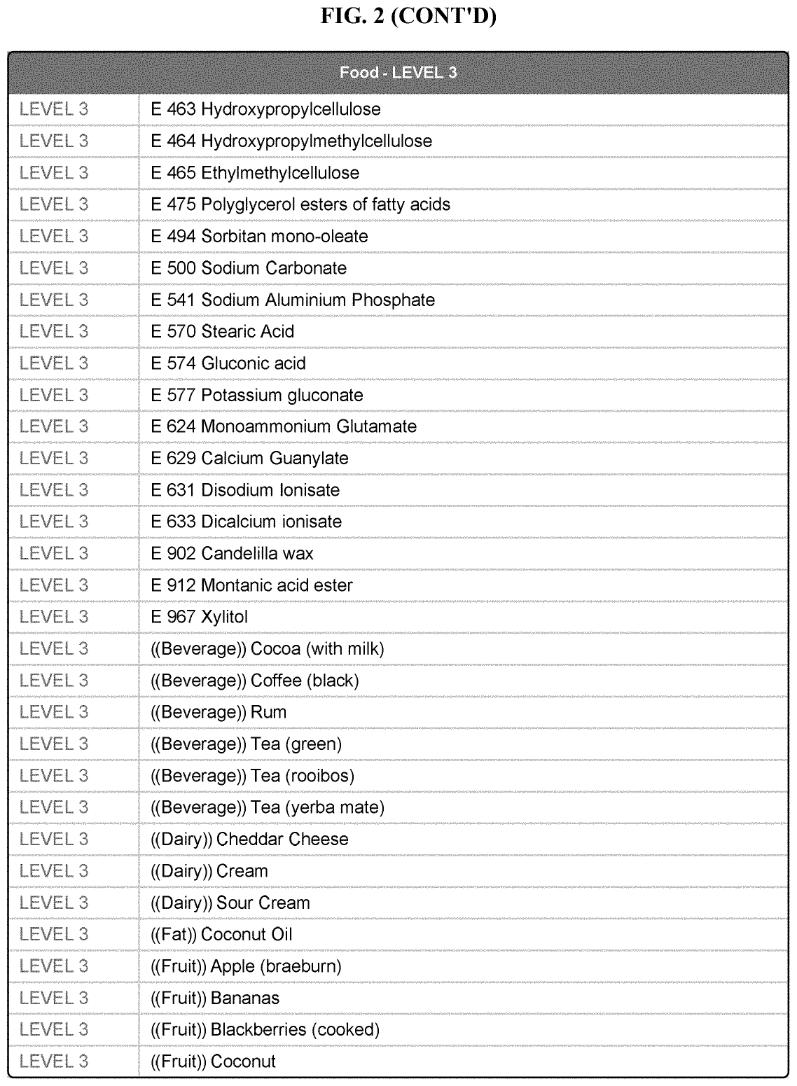

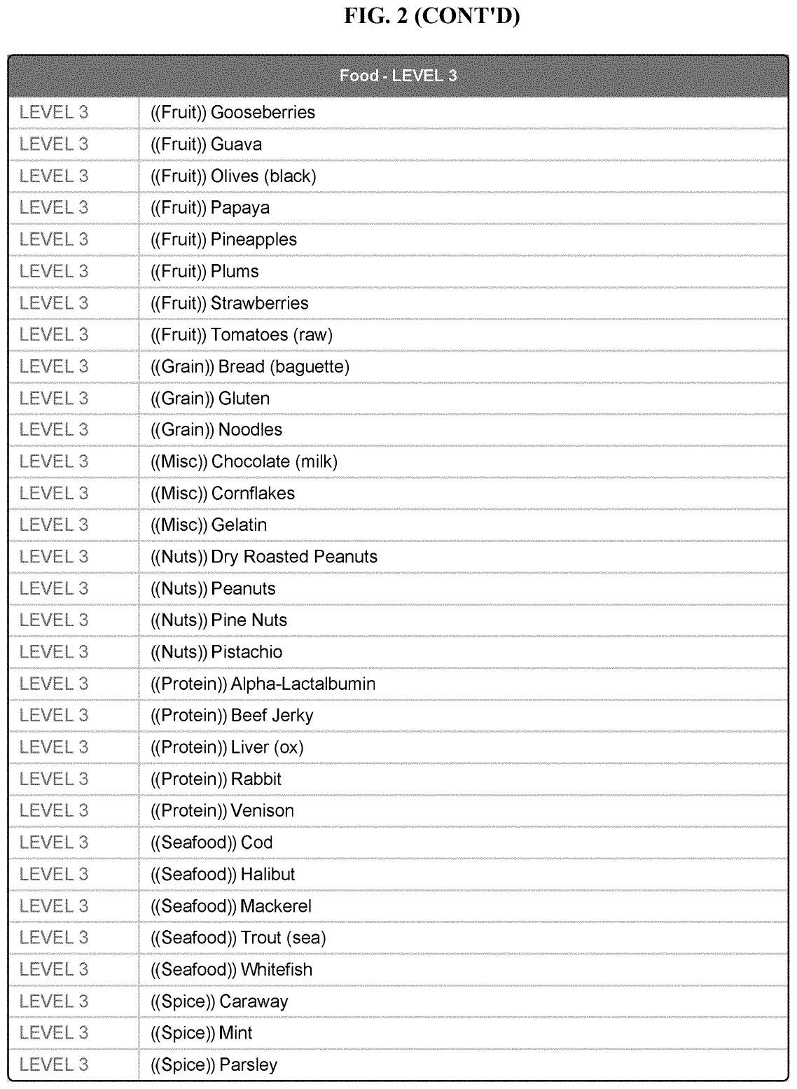

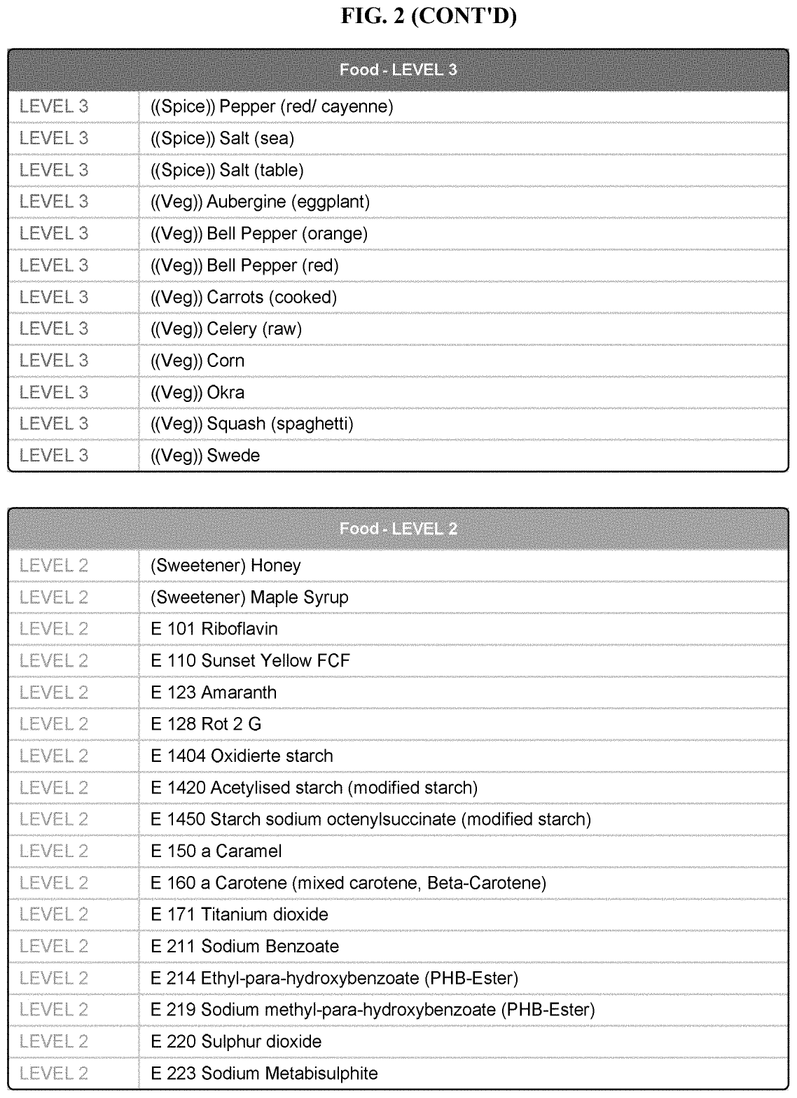

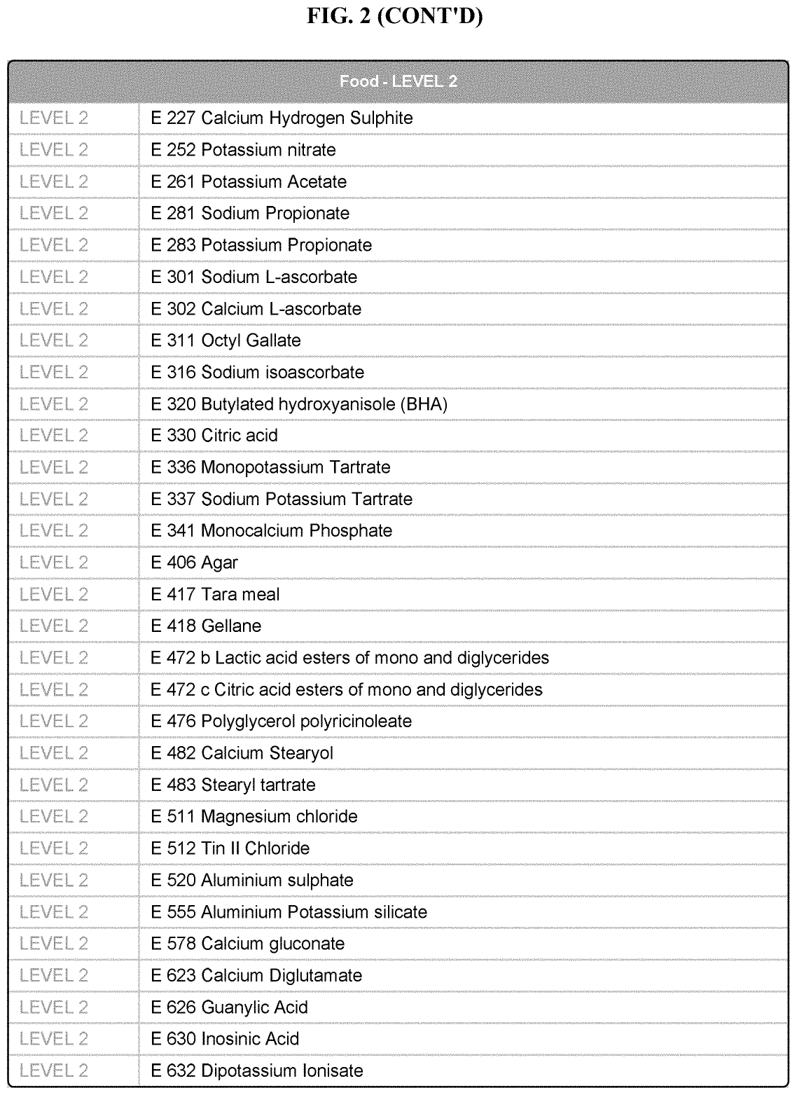

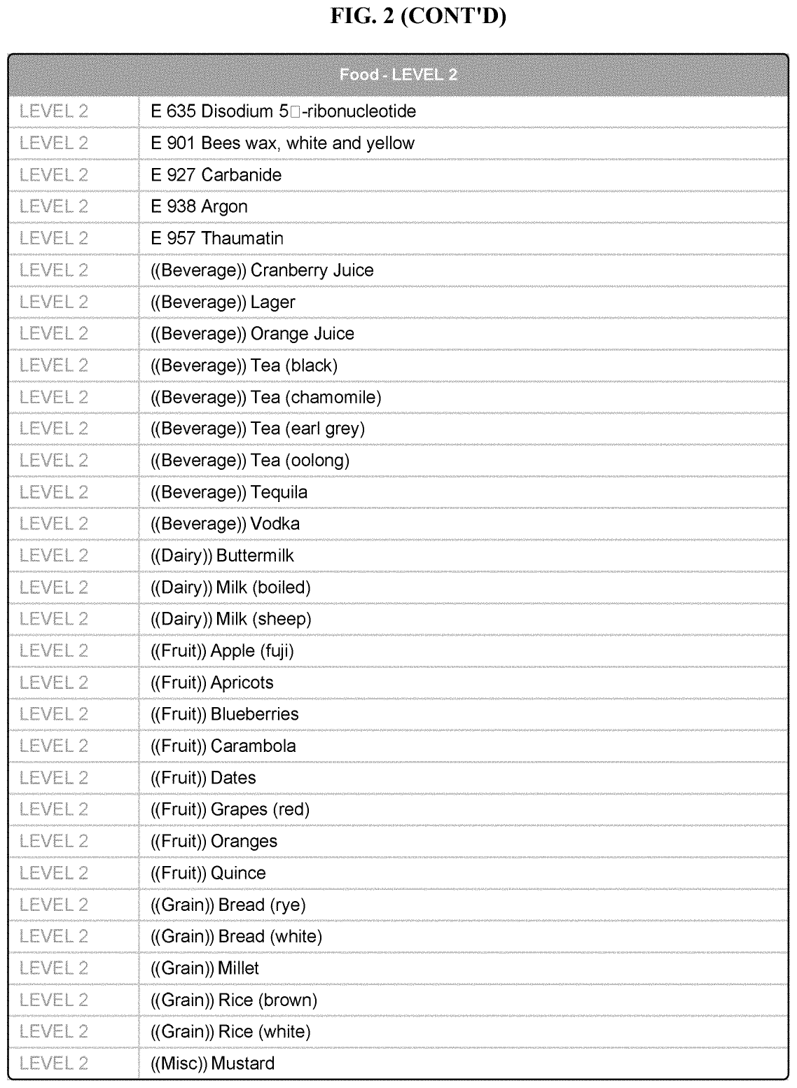

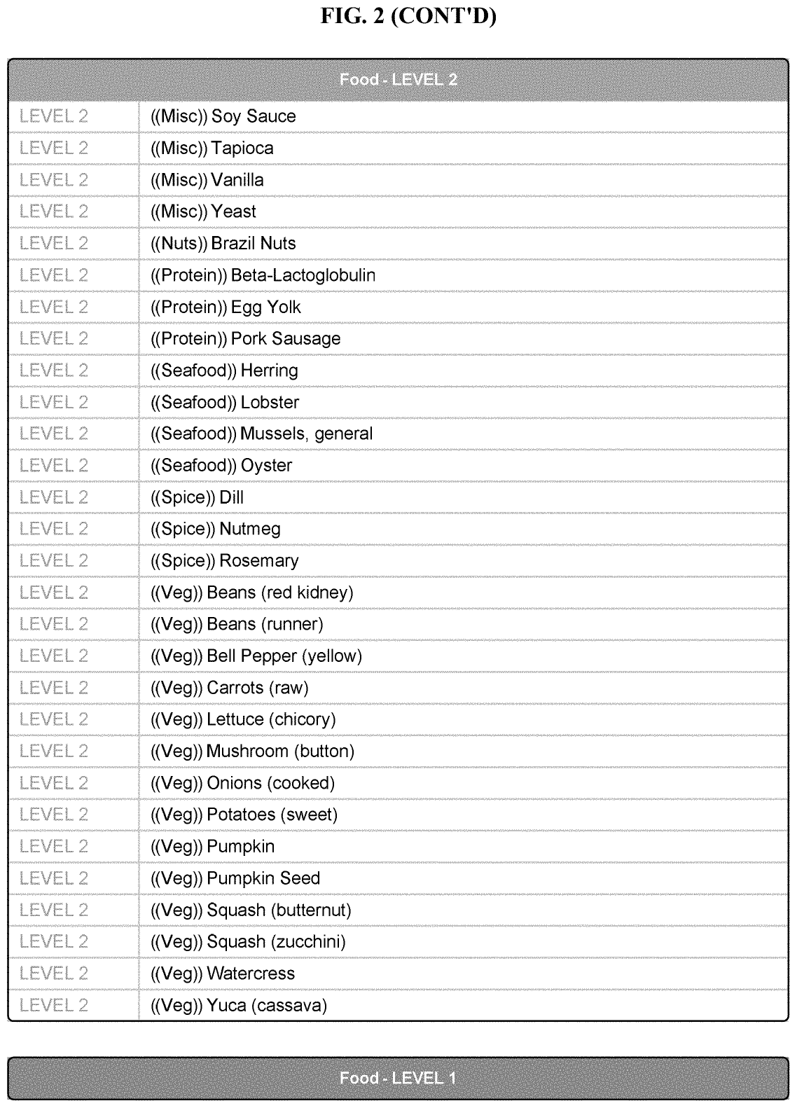

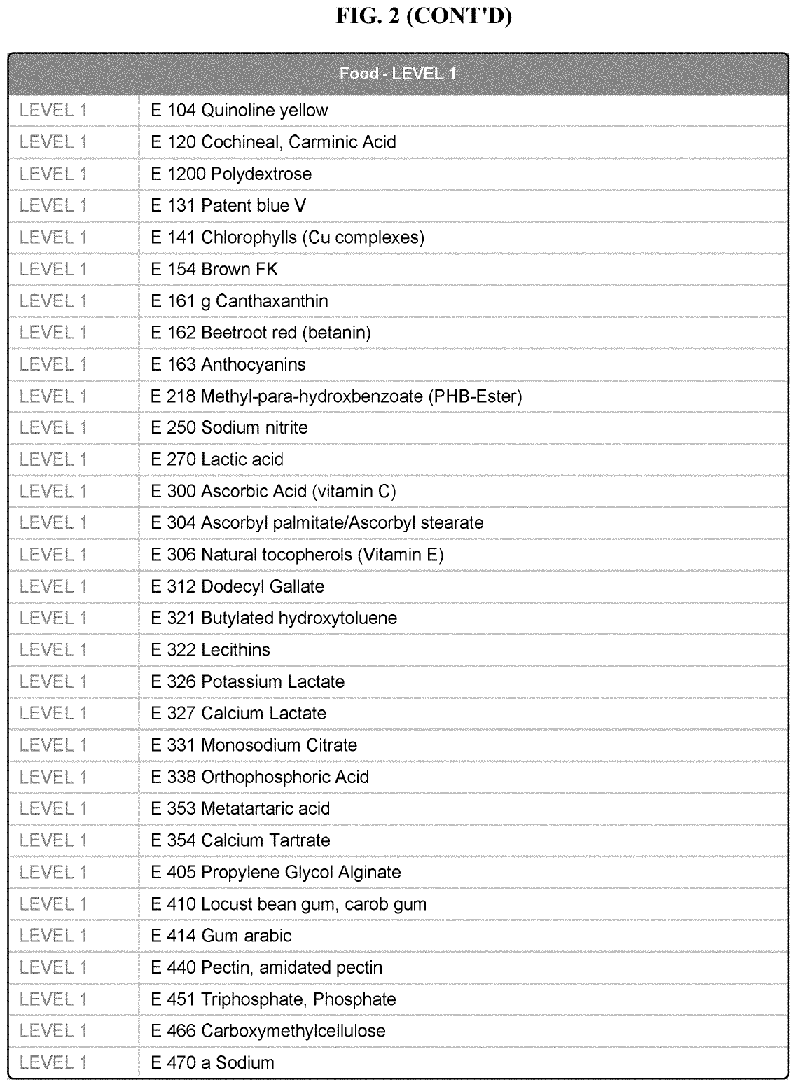

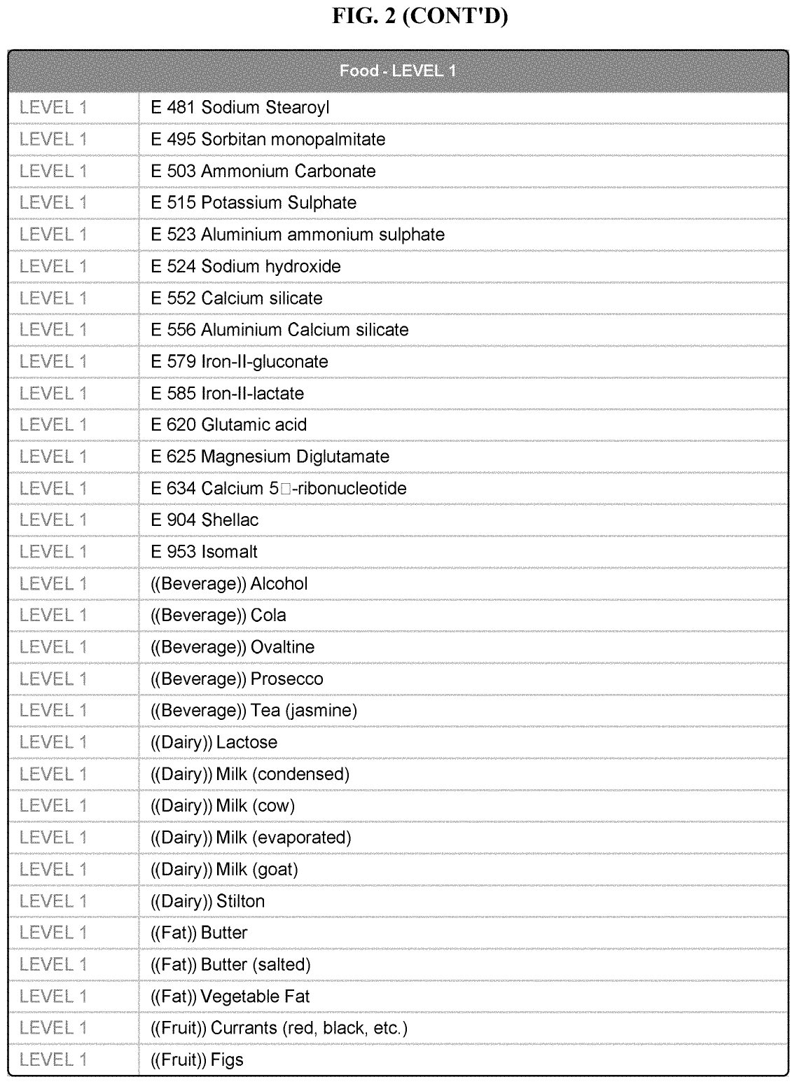

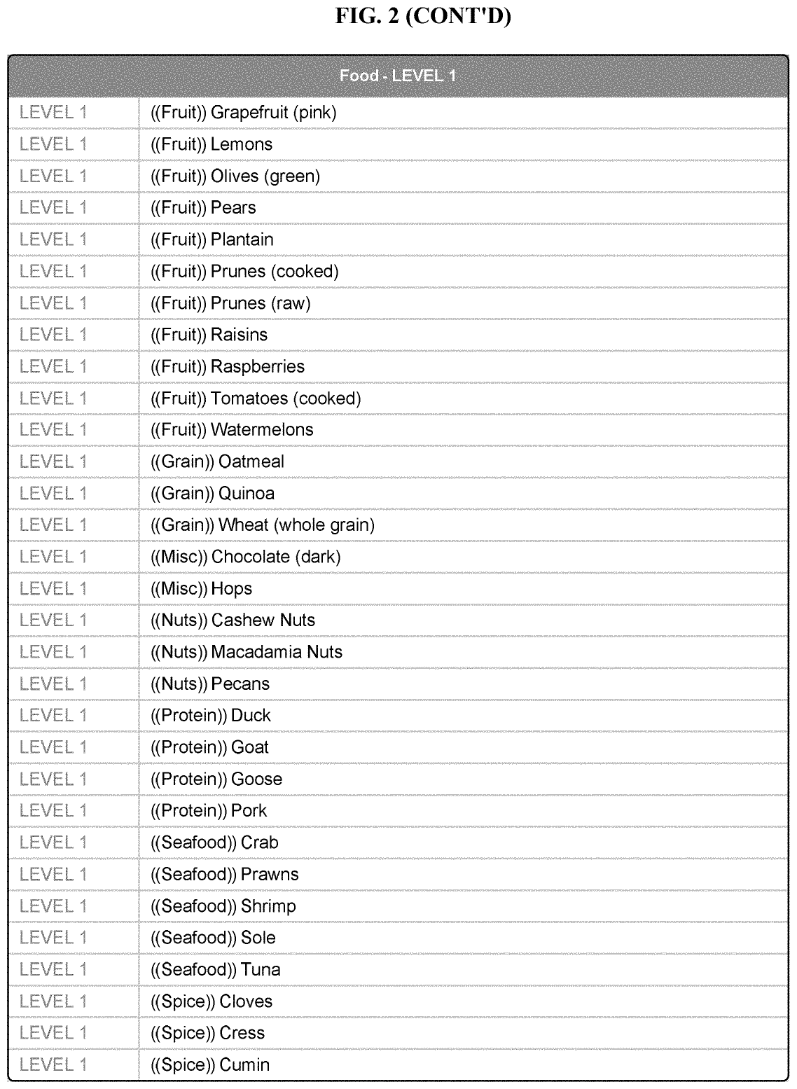

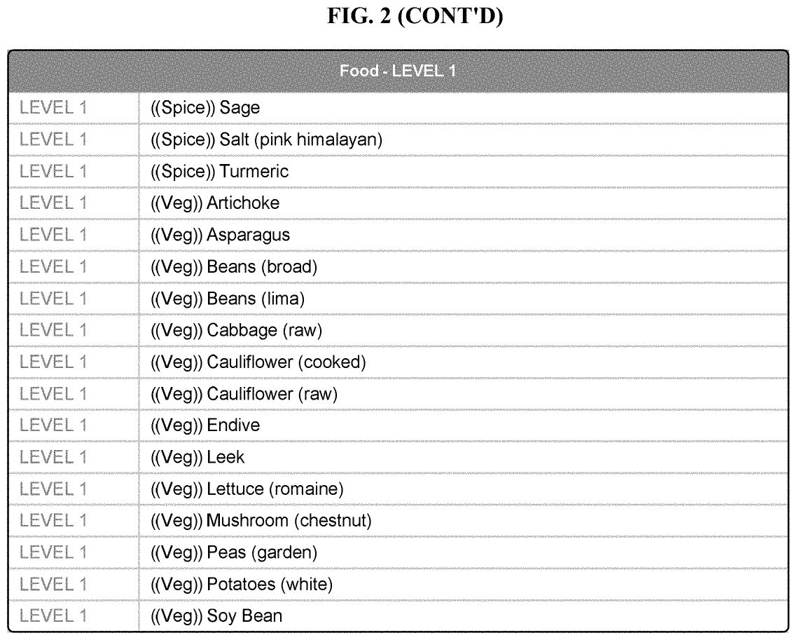

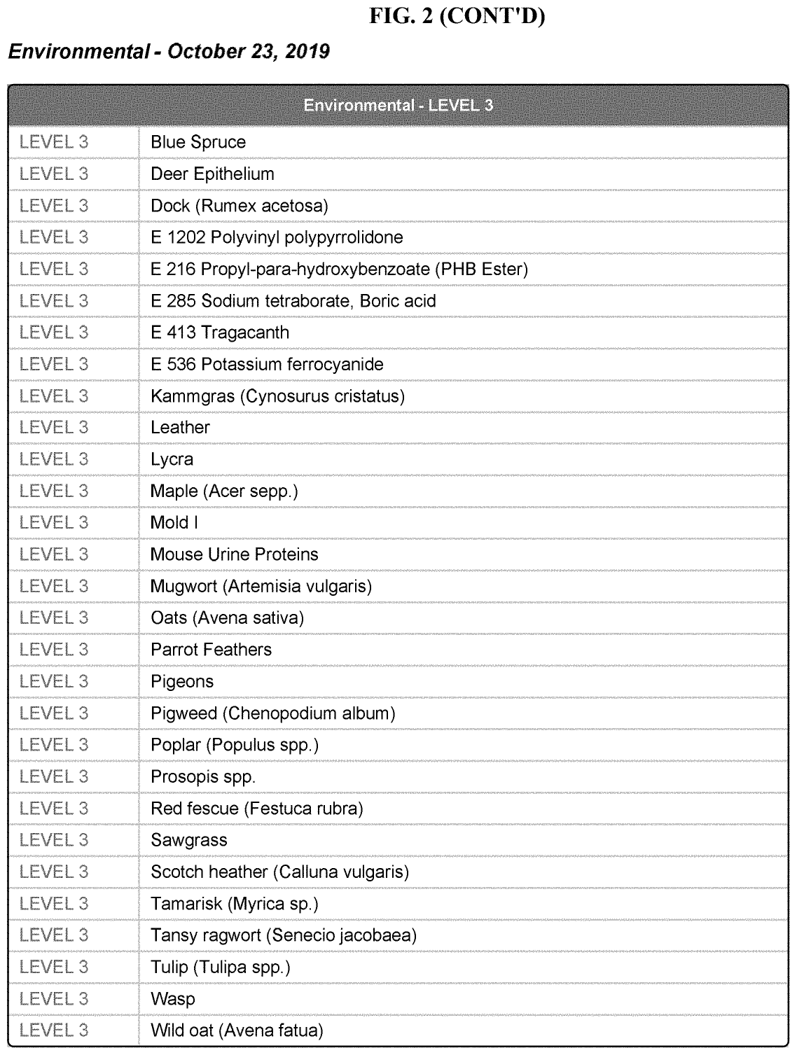

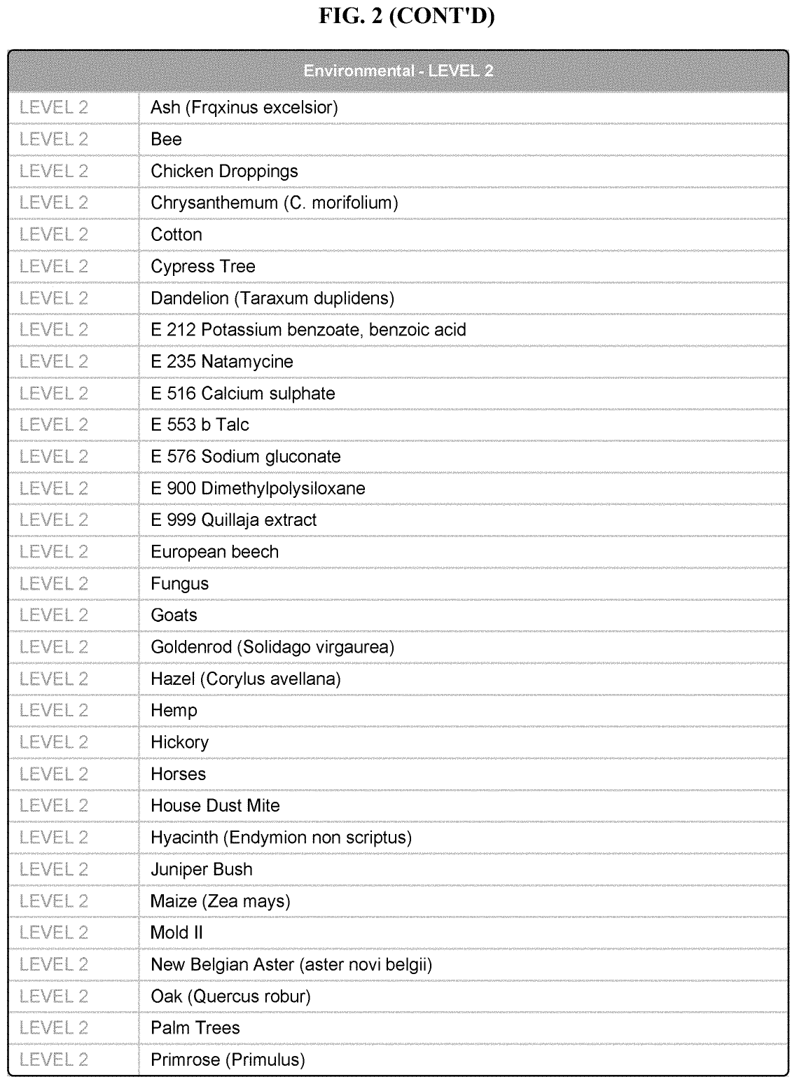

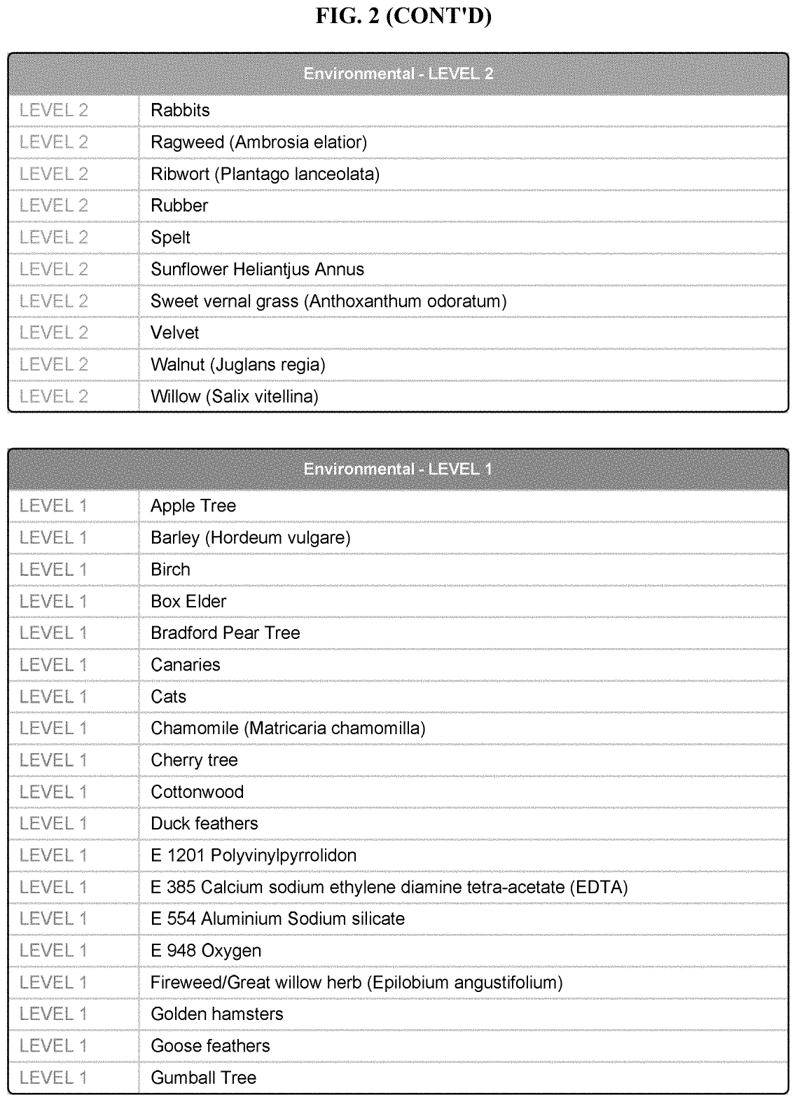

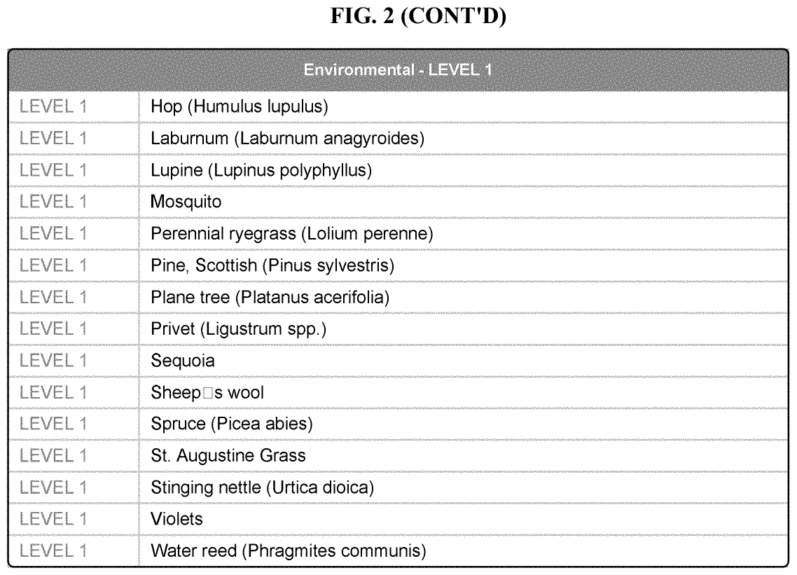

The CRM formats the results into a report that can be sent to the subject (see FIG. 2). The information in the report is specific to the test that was run on the sample. For example, the report for food (food report) lists the intolerances for various item categories, such as protein, dairy, fruit, vegetables, sweeteners, spices, grains, nuts, seafood, and beverages. The food report also lists the level of severity for each item intolerance, such as level 3—most reactive, level 2—moderately reactive, level 1—mildly reactive.

The nutritional deficiency report lists deficiencies for item categories such as essential amino acids, non-essential amino acids, fatty acids, trace minerals, ionic minerals, essential minerals, vitamins, elements, and miscellaneous items, as well as specific items. Items listed with a reported value of −50 to −100 are considered deficient. Specifically, items that register from −50 through −65 are classified and reported as MILD (green color), items that register from −66 through −85 are classified and reported as MODERATE (yellow color), items that register from −86 through −100 are classified and reported as SEVERE (red color).

The metals and minerals report lists any item that the subject has been over exposed to and registers on a scale from 50-100. Items that register from 50-65 are classified and reported as MILD (green color), items that register from 66-85 are classified and reported as MODERATE (yellow color), and items that register from 86-100 are classified and reported as SEVERE (red color). Finally, the environmental report lists every item to which an intolerance was measured, also in levels of severity.

The results are sent to the subject, along with a master list of all the items tested for and an instructional video explaining how to interpret the test results.

While the invention has been described in detail with reference to exemplary embodiments thereof, it will be apparent to one skilled in the art that various changes can be made, and equivalents employed, without departing from the scope of the invention. The foregoing description of the preferred embodiments of the invention has been presented for purposes of illustration and description. It is not intended to be exhaustive or to limit the invention to the precise form disclosed, and modifications and variations are possible in light of the above teachings or may be acquired from practice of the invention. The embodiments were chosen and described in order to explain the principles of the invention and its practical application to enable one skilled in the art to utilize the invention in various embodiments as are suited to the particular use contemplated. It is intended that the scope of the invention be defined by the claims appended hereto, and their equivalents. The entirety of each of the aforementioned documents is incorporated by reference herein.

I claim:

1. A method for determining intolerances to one or more items in a subject, the method comprising:
a) obtaining a sample from the subject;
b) analyzing the sample using a testing machine, wherein said analyzing is selected from the group consisting of bioresonance, radionic, quantum response, and combinations thereof;
c) comparing results of said analyzing to known frequencies of said items; and
d) determining intolerances to one or more items from said comparing step based on degree of frequency difference.

2. The method of claim 1, wherein said one or more items is selected from the group consisting of a food, beverage, nutrient, metal, mineral, environmental factor, and combinations thereof.

3. The method of claim 2, wherein the food comprises an ingredient selected from the group consisting of protein, fruit, dairy, vegetable, sweetener, spice, grain, nut, seafood, and combinations thereof.

4. The method of claim 2, wherein the nutrient comprises an ingredient selected from the group consisting of essential amino acids, non-essential amino acids, fatty acids, trace minerals, ionic minerals, essential minerals, vitamins, elements, and combinations thereof.

5. The method of claim 1, wherein said subject is selected from the group consisting of a human, dog, cat, and horse.

6. The method of claim 5, wherein the subject is a human.

7. The method of claim 1, wherein said sample is hair or fur.

8. The method of claim 1, wherein the testing machine is a multiple analytical resonance system.

9. A kit comprising:
a) a collection container for collecting a sample from a subject;
b) instructions explaining how to collect the sample; and
c) a form to collect information about the subject and determine testing to be conducted on the sample.

10. The kit of claim 9, wherein the container is configured and arranged to hold a sample of hair or fur.

11. The kit of claim 10, wherein the container is configured and arranged to hold a sample from a human.
